(19) United States
(12) Patent Application Publication
(10) Pub. No.: US 2008/0294235 A1
(43) Pub. Date: Nov. 27, 2008

(54) BYPASS GRAFT DEVICE AND DELIVERY SYSTEM AND METHOD

(75) Inventors: Eran Bendory, Tel Aviv (IL); Ehud Bendory, Tel Aviv (IL); Ido Kilemnik, Herzlia (IL)

Correspondence Address: HESLIN ROTHENBERG FARLEY & MESITI PC, 5 COLUMBIA CIRCLE, ALBANY, NY, 12203, US

(73) Assignee: INVERTHIS LTD., Ness Ziona (IL)

(21) Appl. No.: 12/030,327

(22) Filed: Feb. 13, 2008

Related U.S. Application Data
(60) Provisional application No. 60/939,862, filed on May 24, 2007.

(51) Int. Cl. A61F 2/06

(52) U.S. Cl. 623/1.13; 623/1.23; 623/1.35

(57) ABSTRACT

A device, system and methods for a minimally invasive bypass procedure, includes a graft having a supporting segment, an extending segment and a bypass segment. The supporting segment includes a flexible portion and a supporting member, and the extending segment includes a flexible portion and a supporting member. The extending segment has an initial configuration wherein it is unextended and a final configuration wherein it is extended proximally into the vessel. The bypass segment is positioned outside of the vessel to be treated, and the supporting segment is positioned within the vessel and distal to the incision. Once the supporting segment and the supporting member are anchored in place, the extending segment is extended proximally past the incision site. Thus, the supporting segment and the extending segment are substantially aligned, and allow for blood flow through the vessel while also providing blood flow around an obstruction via the bypass segment.

### FIELD OF THE INVENTION

The present invention relates to bypass grafts, and more particularly to a bypass graft device and system which can be inserted via a minimally invasive technique.

### BACKGROUND OF THE INVENTION

At times it is necessary to perform a bypass procedure when a vessel is occluded. These types of procedures are generally performed using autografts, allografts, xenografts or synthetic grafts, wherein one end of the graft is attached to an end of the occluded vessel which is proximal to the occlusion, and the other end of the graft is attached to a portion of the occluded vessel which is distal to the occlusion, thereby bypassing the occluded portion of the vessel. This type of procedure generally requires surgical access to both sites (ie, the area of the occluded vessel proximal to the occlusion and the area of the occluded vessel distal to the occlusion).

In order to position a graft via a minimally invasive procedure, it is necessary to find a way to anchor the graft in the vessel without the need for suturing or other attachment means typically used in surgical procedures.

Generally, devices have been developed for grafting wherein the graft is to provide an access path through an area having an aneurysm or blocked vessel. These types of grafts are generally inserted through a blood vessel which may be remote from the site of the aneurysm or blocked vessel. However, graft devices for bypass wherein the bypass site is accessed directly are not currently available.

Several graft devices and methods for treating an aneurysm or blockage are disclosed.

A graft having a branched side tube which can be inverted is disclosed in U.S. Pat. No. 6,814,747 to Anson et al. The graft/stent disclosed therein includes a plurality of ring-like rigid members having a contracted shape and an expanded shape. The inverted portion is re-inverted by pulling on a cord. A disadvantage of this design is that in order to pull on the cord, the cord must be accessed from the other side, requiring an additional access site.

A single-piece bifurcated graft for insertion into the aorta is disclosed in U.S. Pat. No. 5,904,713 to Leschinsky. The graft has an inverted portion and no central section. Two bifurcated sections are joined at the top. Upon introduction, the graft is attached to the wall of the vessel in the middle section, and the inverted portion is un-inverted. Once in place, the two sections are roughly parallel.

An intraluminal prosthesis is disclosed in U.S. Pat. No. 6,016,810 to Ravenscroft. The prosthesis includes a tubular, flexible graft having a proximal open end, and at least one distal open end terminating in a hem. The hem is inverted so that it is disposed as a cuff within the graft. Upon withdrawing the distal open end from inside the cuff, the cuff will unfold. The hem may be folded a second time to form a second cuff within the graft. Although deployment of the inverted portion may be accomplished by pushing rather than pulling, the additional folds in the graft material would likely result in a relatively large overall diameter for the system.

The devices described above are used for providing a passage of blood in an area where blood flow may be compromised to due an aneurysm or blockage. A device used for bypass is disclosed in U.S. Pat. No. 6,575,168 to LaFontaine et al. The graft disclosed therein is used by making an incision in the aorta and an incision in a second vessel. The graft section is inverted and pushed through a coupler to reach the second vessel. The first end of the graft section may be anchored in the aorta via a stent, while the second end of the graft section is anchored by other means, such as adhesive, for example.

It would be advantageous to have a device, system and method for a minimally invasive bypass procedure, which is small enough in diameter to be placed in relatively small vessels such as the femoral artery, which can be easily anchored in place, and which can be deployed without separately accessing another end of the device.

### SUMMARY OF THE INVENTION

In accordance with embodiments of the present invention, there is provided a device for positioning in a vessel. The device includes a supporting segment comprised of a flexible material having a substantially tubular configuration, a supporting member comprised of a substantially rigid material, the supporting segment having a supporting segment proximal end and a supporting segment distal end, an extending segment having a substantially tubular configuration including a flexible inverting portion comprised of a flexible material having a proximal end and a distal end, and an internal supporting member comprised of a substantially rigid material having a proximal end and a distal end. The internal supporting member proximal end is attached to the flexible inverting portion proximal end at an attachment area, wherein in an initial configuration, the flexible inverting portion is inverted such that the flexible inverting portion proximal end is distal to the flexible inverting portion distal end and the internal supporting member is distal to the flexible inverting portion, and wherein in a deployed configuration, the flexible inverting portion is un-inverted such that the flexible inverting portion proximal end is proximal to the flexible inverting portion distal end and the internal supporting member is positioned within the flexible inverting portion. The device further includes a bypass segment comprised of a flexible material having a substantially tubular configuration including a proximal end and a distal end, wherein the supporting segment, the inverting segment and the bypass segment are in fluid communication with one another, and wherein the supporting segment proximal end, the extending segment distal end and the bypass segment proximal end are connected at a connecting area.

In accordance with additional embodiments of the present invention, there is provided a method for performing a minimally invasive bypass procedure. The method includes providing a device having a supporting segment, an extending segment, and a bypass segment in fluid communication with one another and wherein the extending segment is initially in an unextended configuration, making an incision in a vessel to be treated, inserting the supporting segment directly into the vessel through the incision and positioning the supporting segment in the vessel distal to the incision with the bypass segment positioned through the incision and out of the vessel, anchoring the supporting segment into the vessel, and extending the extending segment in a proximal direction such that the extending segment is positioned in the vessel proximal to the incision.

In accordance with yet additional embodiments of the present invention, there is provided a delivery system for delivery of a graft to a vessel. The delivery system includes a guidewire having a proximal end and a distal end, wherein the proximal end is positionable outside of a body and wherein the distal end is configured to enter the body at an incision site, the distal end having a bent configuration. The guidewire further includes a proximal extension portion extending proximally from the distal end. The delivery system further includes an internal sheath having a first portion for enclosing a first segment of the graft and a second portion for enclosing at least a portion of the guidewire and extending proximally to a point outside of the body, wherein the proximal extension portion of the guidewire is partially enclosed by the first portion of the internal sheath and is removably attached to the first segment of the graft located within the internal sheath, the internal sheath movable with respect to the guidewire, a stopper attached to the proximal extension portion of the guidewire for holding the graft in place while the internal sheath is removed, and an external sheath for enclosing a second member of the graft and extending proximally to a point outside of the body, the external sheath movable with respect to the internal sheath and the guidewire.

In accordance with yet additional embodiments of the present invention, there is provided a device for positioning in a vessel. The device includes a supporting segment configured to be placed directly through an incision in a vessel and to be anchored into the vessel in an area distal to the incision, an extending segment, wherein in a first configuration the extending segment is positioned within the supporting segment and in a second configuration the extending segment is extended proximally so as to be anchored into the vessel in an area proximal to the incision, and a bypass segment in fluid communication with the supporting segment and the extending segment, the bypass segment positioned through the incision and outside of the vessel.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1 is a schematic illustration of a vessel having a bifurcation and a blockage in one of the branched vessels;

FIGS. 2A-2D are schematic illustrations of a device in accordance with embodiments of the present invention for treating a blocked vessel such as that depicted in FIG. 1;

FIG. 3A-3C are schematic perspective illustrations of the device of FIG. 2, in accordance with embodiments of the present invention;

FIG. 4A-4E are illustrations of a delivery system for the device of FIGS. 2 and 3, in accordance with embodiments of the present invention;

FIGS. 5A-5F are cross-sectional illustrations of the device and delivery system of FIGS. 2-4, in various stages of deployment; and

FIGS. 6A-6H are illustrations of the various stages of deployment as described with reference to FIGS. 5A-5F, shown in the vessel.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention is directed to a device, system, and methods for positioning of a bypass graft. The principles and operation of a device, system and methods according to the present invention may be better understood with reference to the drawings and accompanying descriptions.

Before explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

For the purposes of the present application, the terms “distal” and “proximal” refer to the orientation of the device within the body of a patient. As used herein, “distal” refers to the end of the device extended into the body first, and “proximal” refers to the end of the device located farthest from the distal end of the device when the device is in its fully deployed configuration. The term “incision” refers to any opening of any size in the body.

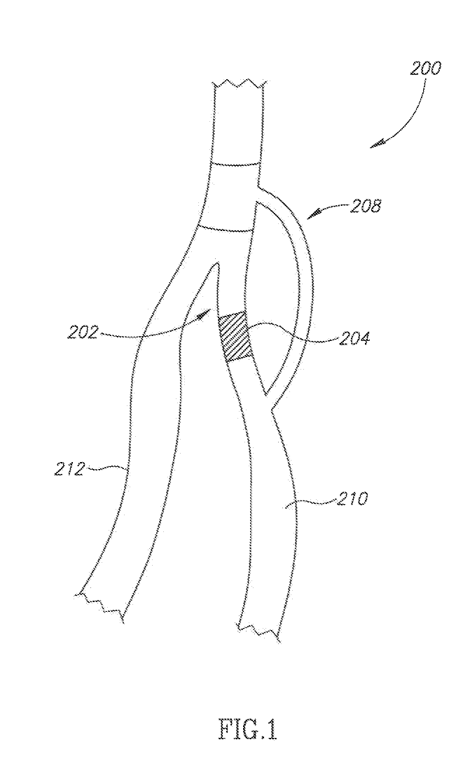

Reference is now made to FIG. 1, which is a schematic illustration of a vessel 200 having a bifurcation 202. In one example, vessel 200 is a femoral artery, which branches off into a superficial femoral artery 210 and a deep femoral artery 212. In other embodiments, vessel 200 may be another vessel in the body with branches. A blockage 204 is located in one of the branch vessels (in the present embodiment, the superficial femoral artery 210). Current methods for bypassing blockage 204 rely on surgical bypass methods, which may include autografts, xenografts or artificial grafts. It is a feature of the present invention to provide a minimally invasive method and system for delivery of a bypass device 208 into a vessel such as the femoral artery without the need for an open surgical procedure.

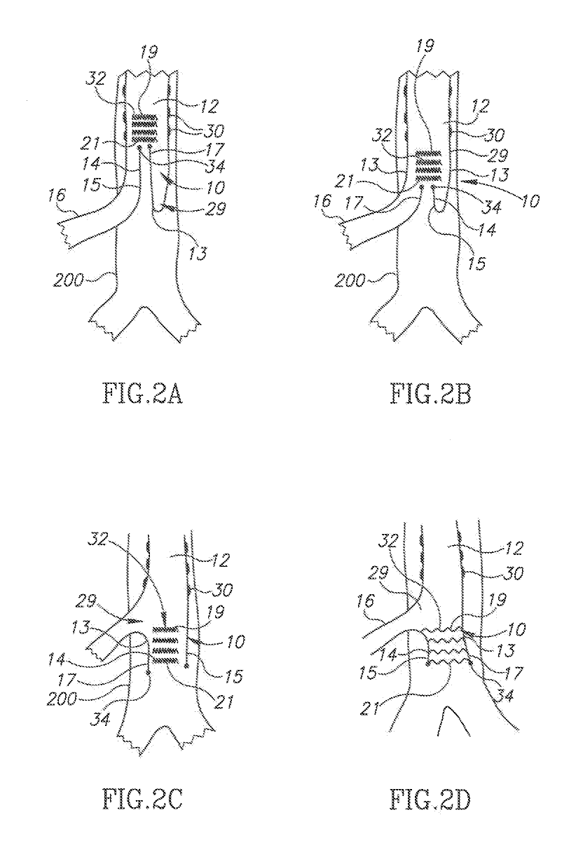

Reference is now made to FIGS. 2A-2D, which are schematic illustrations of a device 10 in accordance with embodiments of the present invention. The main operating principles of device 10 are depicted in FIGS. 2A-2D, but the details of device 10 and systems and methods for its delivery into vessel 200 are described further hereinbelow. Device 10 includes a supporting segment 12, an extending segment 14, and a bypass segment 16. Supporting segment 12, extending segment 14 and bypass segment 16 are comprised of flexible graft material. In some embodiments, each of supporting segment 12, extending segment 14 and bypass segment 16 is comprised of the same flexible material. In other embodiments, one or more of the segments are comprised of different flexible materials. Flexible materials may be any suitable flexible material such as, but not limited to, Dacron, PTFE, fluoro-based compounds, biological materials, etc. Supporting segment 12 has a supporting member 30 which provides support to the flexible material of supporting segment 12. In some embodiments, supporting member 30 is an external supporting member and surrounds the flexible material. In other embodiments, supporting member 30 is an internal supporting member positioned internally with respect to the flexible material. In yet additional embodiments, supporting member is sandwiched between two layers of flexible material. Supporting member 30 is comprised of a substantially rigid material for support, such as a metal or a hard polymer, for example. Supporting member 30 may be a stent, for example. Similarly, extending segment 14 has a flexible inverting portion 15 and an internal supporting member 32, wherein internal supporting member 32 is comprised of substantially rigid material. The material comprising internal supporting member 32 may be the same as or different than the material comprising supporting member 30. In some embodiments, internal supporting member 32 further comprises an additional layer of flexible material therein, wherein said internal supporting member 32 surrounds the additional layer of flexible material so that blood flowing through extending segment 14 may be in contact with a flexible material rather than a substantially rigid material.

Flexible inverting portion 15 has a proximal end 17 and a distal end 13, and internal supporting member 32 has a proximal end 21 and a distal end 19. Flexible inverting portion 15 is attached to supporting segment 12 at its distal end 13 at a connecting area 29, and proximal end 21 of internal supporting member 32 is attached to proximal end 17 of flexible inverting portion 15 at an attachment area 34. In the embodiment shown in FIGS. 2A-2D, initially, extending segment 14 is in an inverted configuration, as shown in FIG. 2A, with flexible inverting portion 15 proximal to extending segment proximal end 17.

As shown in FIGS. 2B and 2C, when deploying extending segment 14, only flexible inverting portion 15 is inverted, while internal supporting member 32 remains in its initial configuration. In this way, distal end 13 of flexible inverting portion 15 retains a distal position while proximal end 17 of flexible inverting portion 15 moves from a distal position to a proximal position. During these movements, internal supporting member 32 remains in its initial configuration, such that distal end 19 of internal supporting member 32 begins to align with distal end 13 of flexible inverting portion 15, while proximal end 21 of internal supporting member 32 begins to align with proximal end 17 of flexible inverting portion 15. Internal supporting member 32 sits within extending segment 14 and acts as a support for flexible inverting portion 15. In this way, the substantially rigid members do not need to undergo deformation, un-inversion or other complicated configurations. In some embodiments, internal supporting member 32 is crimped onto a balloon, and in a final step, the balloon is expanded, thereby expanding internal supporting member 32, as shown in FIG. 2D. In other embodiments, internal supporting member 32 is enclosed within an outer sheath, which is then removed upon deployment of internal supporting member 32. A bypass segment 16 remains positioned at an insertion site throughout the procedure, as will be described in greater detail.

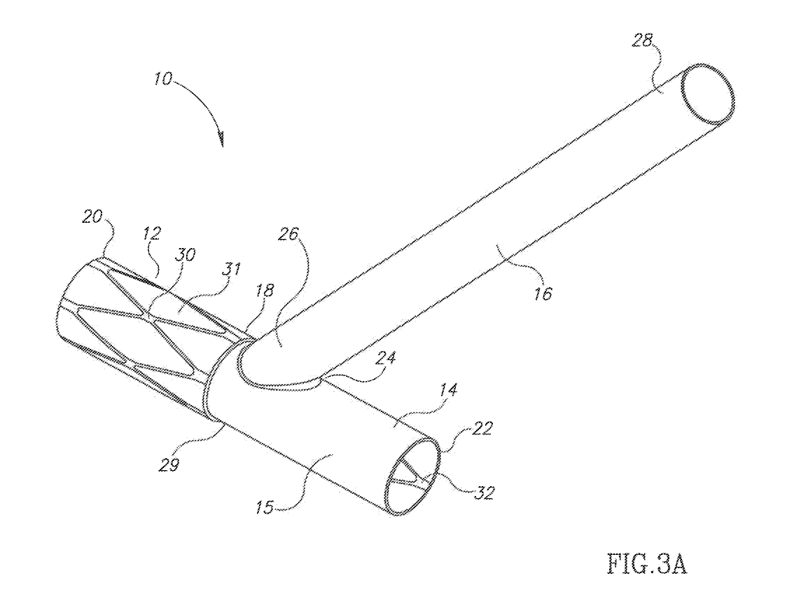

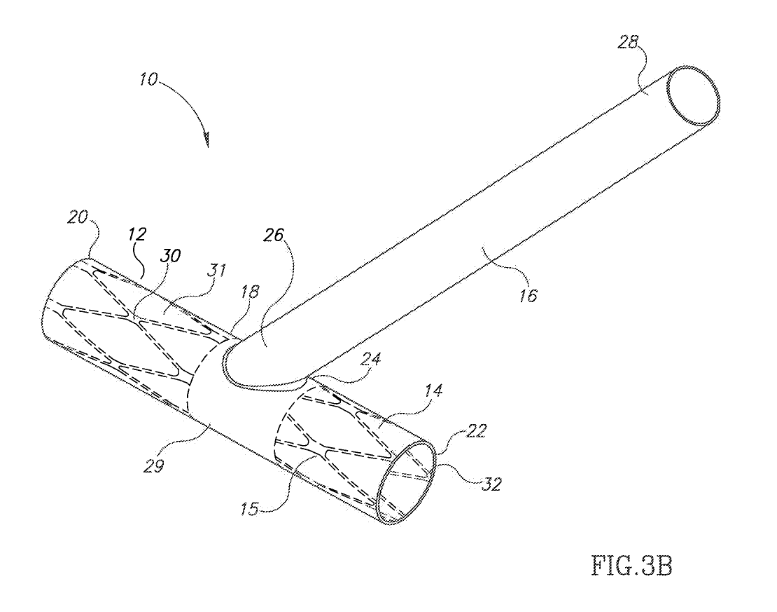

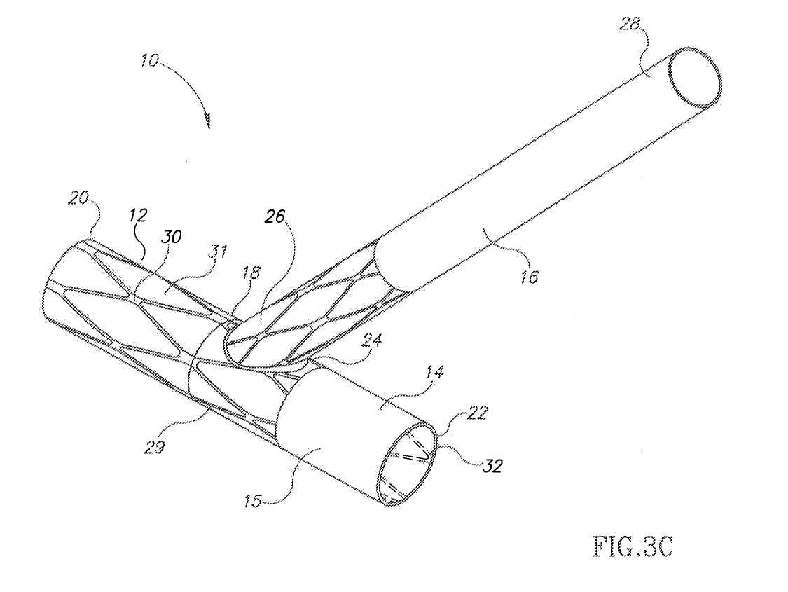

Reference is now made to FIGS. 3A-3C, which are schematic perspective illustrations of device 10, in accordance with embodiments of the present invention. Device 10 may be described as having three segments: a supporting segment 12, an extending segment 14 and a bypass segment 16, wherein supporting segment 12 and extending segment 14 are designed to be positioned within a vessel, and wherein bypass segment 16 is a synthetic bypass material which is attached to supporting segment 12 and extending segment 14 and which is designed to provide a bypass area for blood flow. An intersection of supporting segment 12, extending segment 14 and bypass segment occurs at connecting area 29. Supporting segment 12 has a supporting segment proximal end 18, which is attached to connecting area 29, and further includes a supporting segment distal end 20, configured to enter the vessel first. Extending segment 14 has an extending segment proximal end 22, which is the end of extending segment 14 furthest from supporting segment distal end 20, and an extending segment distal end 24, which is attached to connecting area 29. Bypass segment 16 has a bypass segment proximal end 28 which is the end of bypass segment 16 which is furthest from supporting segment distal end 20, and a bypass segment distal end 26, which is adjacent to connecting area 29. Supporting segment proximal end 18 and extending segment distal end 24 are positioned adjacent to bypass segment proximal end 26 at connecting area 29. Supporting segment 12 includes a supporting member 30 and a flexible portion 31, wherein in some embodiments, supporting member 30 is positioned external to flexible portion 31, as depicted in FIG. 3A, and in other embodiments, supporting member 30 is positioned internal to flexible portion 31. In additional embodiments, as shown in FIG. 3B, two layers of flexible portion 31 may be used, with supporting member 30 sandwiched in between the two layers, as depicted by dotted lines. Extending segment 14 includes a flexible inverting portion 15 and an internal supporting member 32. In an initial configuration, extending segment 14 is inverted, or folded or rolled, into device 10 such that extending segment distal end 24 is proximal to extending segment proximal end 22. After full deployment of device 10 within the vessel, the un-inverted configuration shown in FIGS. 3A-3C is obtained. In some embodiments, an internal surface of internal supporting member 30 may be covered with another layer of flexible material, as shown in FIG. 3B. In some embodiments, supporting member 30 extends over connecting area 29, and in additional embodiments may extend even further over a portion of bypass segment 16, as shown in FIG. 3C.

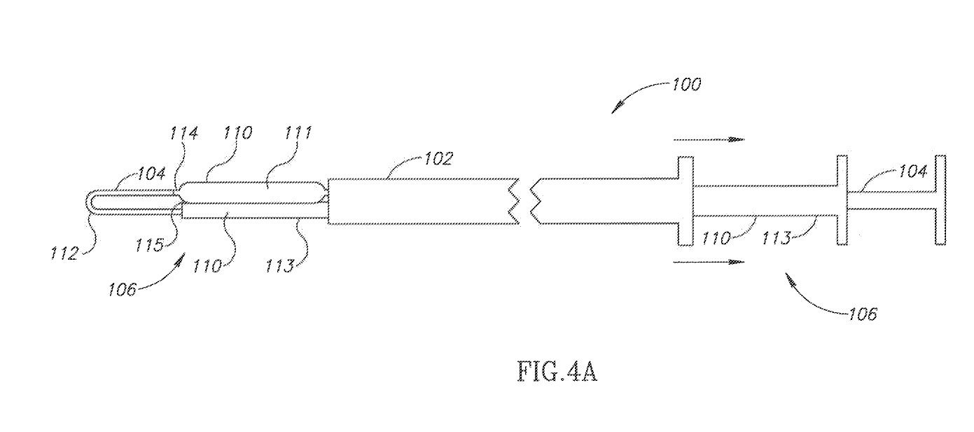

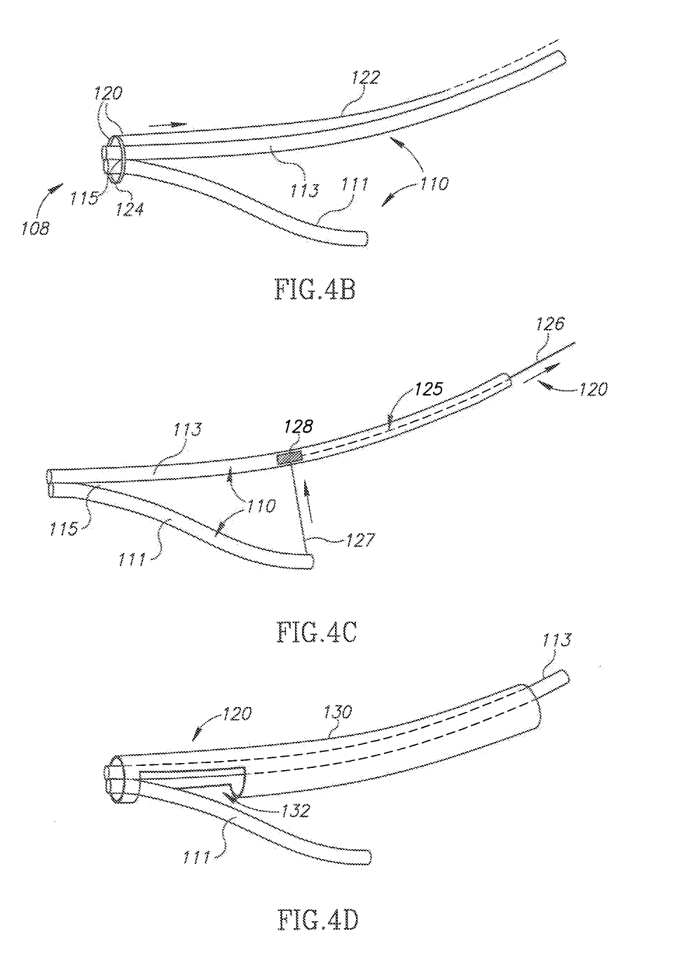

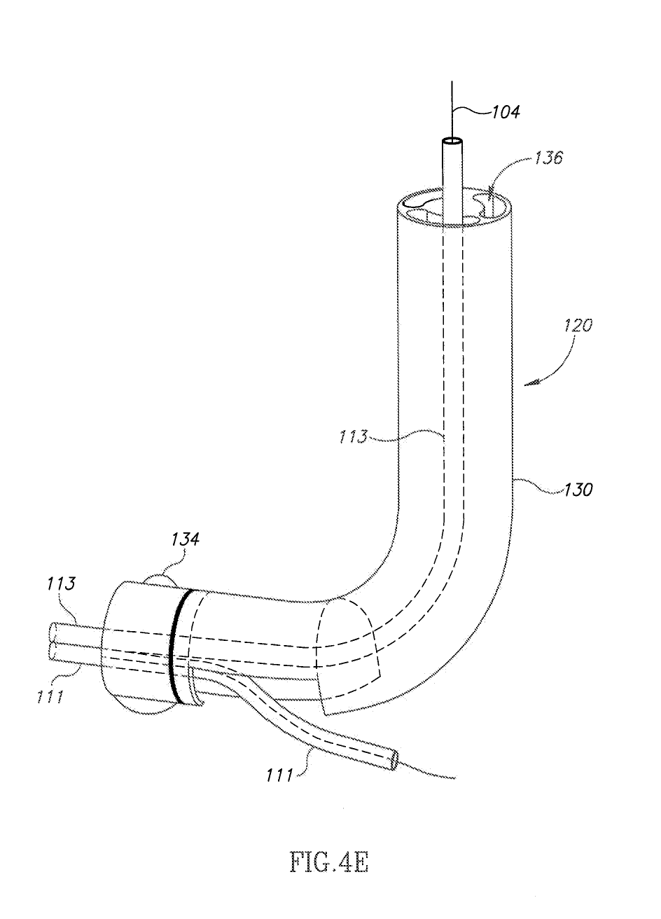

Reference is now made to FIG. 4A, which is an illustration of a delivery system 100 for device 10, in accordance with one embodiment of the present invention. Delivery system 100 has a supporting segment sheath 102 surrounding supporting segment 12 and extending proximally to a delivery system proximal end 106. Supporting segment sheath 102 may initially be positioned over the entire distal portion of device 10, and may then be moved proximally so as to release supporting segment 12. Supporting segment sheath 102 is depicted in FIG. 4A as being partially pulled back proximally, exposing an internal sheath 110. Internal sheath 110 has an internal supporting member sheath portion 111 and a guidewire sheath portion 113. Internal supporting member sheath portion 111 and guidewire sheath portion 113 are attached to one another at their respective distal ends at an attachment area 115 and are configured to separate from one another proximal to attachment area 115. Means for rejoining internal supporting member sheath portion 111 and guidewire sheath portion 113 proximal to attachment area 115 after they have been separated from one another (i.e. after deployment of device 10) are described in greater detail with reference to FIGS. 4B-4E. A guidewire 104 extends from proximal end 106 (proximal to the proximal end of supporting segment sheath 102) to a delivery system distal end 108 and is enclosed within guidewire sheath portion 113 of internal sheath 110. At delivery system distal end 108, guidewire 104 has a bent distal portion 112, and further includes a proximal extension portion 114 which is attached to internal supporting member 32, positioned within internal supporting member sheath portion 111 of internal sheath 110. In some embodiments, bent distal portion 112 is external to internal sheath 110. Internal supporting member sheath portion 111 of internal sheath 110 extends from an area proximal to bent distal portion 112 and covers internal supporting member 32. Guidewire sheath portion 113 extends from an area proximal to bent distal portion proximally until proximal end 106 of delivery system 100. Guidewire sheath portion 113 is moveable with respect to guidewire 104. Since internal supporting member sheath portion 111 and guidewire sheath portion 113 are attached to one another, moving guidewire sheath portion 113 results in simultaneous movement of internal supporting member sheath portion 111.

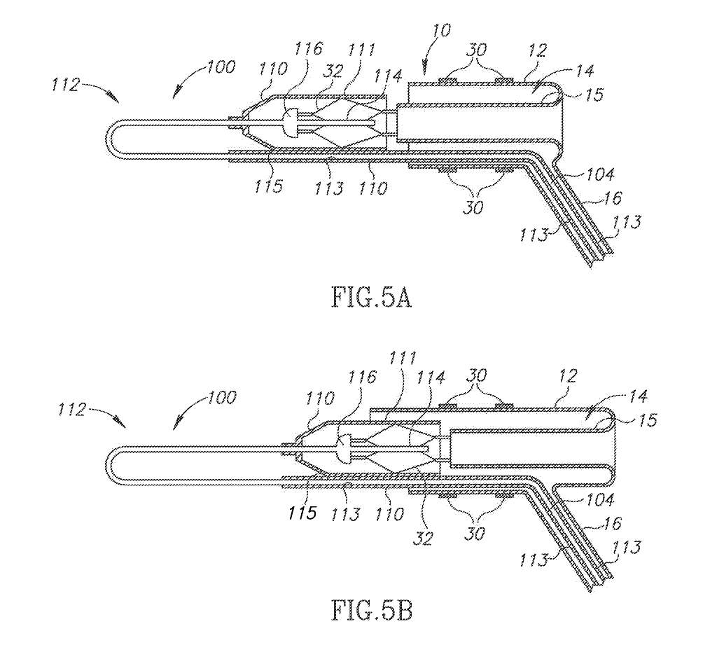

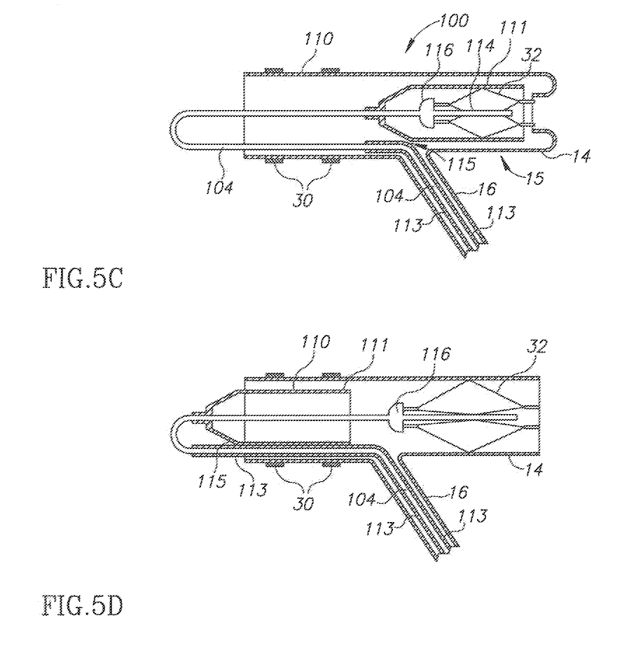

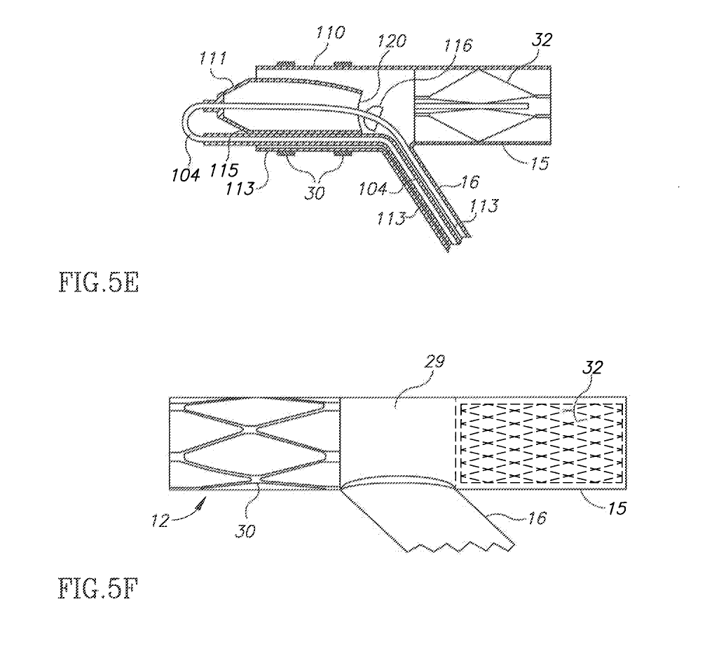

Reference is now made to FIGS. 4B-4E, which are illustrations of internal sheath 110, showing internal supporting member sheath portion 111 and guidewire sheath portion 113 separated from one another proximal to attachment area 115. This separation allows for deployment of device 10 within the vessel, as shown below with respect to FIG. 5C. However, in order to retract internal sheath 110 after deployment of device 10, it is necessary to rejoin internal supporting member sheath portion 111 and guidewire sheath portion 113 to enable both of these portions of internal sheath 110 to be retracted from the vessel together. Thus, a linking element 120 may be included for this purpose.

In one embodiment, as shown in FIG. 4B, linking element 120 includes a wire 122 extending from proximal end 106 of system 100 to distal end 108 of system 100 external to internal sheath 110, with a wire loop portion 124 at distal end 108. Wire loop portion 124 is configured to surround internal sheath 110. By pulling wire 122 proximally, internal supporting member sheath portion 111 and guidewire sheath portion 113 are brought into contact with one another. Once they are in contact, wire 122 and internal sheath 110 may be pulled together proximally to remove them both from the vessel.

In another embodiment, as shown in FIG. 4C, linking element 120 includes a cable 126 having an internal portion 125 and an attachment portion 127. Internal portion 125 is positioned through guidewire sheath portion 113 of internal sheath 110 and extends distally through guidewire sheath portion 113 until it reaches an opening 128. Attachment portion 127 of cable 126 is a portion of cable 126 which extends through opening 128 and is attached to internal supporting member sheath portion 111. Thus, by pulling proximally on cable 126, internal supporting member sheath portion 111 is brought into contact with guidewire sheath portion 113. Once they are in contact, cable 126 and internal sheath 110 may be pulled together proximally to remove them both from the vessel. Cable 126 may be of any suitable configuration, including a wire, a rope, a string, or any other relatively flexible material which is suitable for the method described herein.

In another embodiment, as shown in FIG. 4D, linking element 120 includes an attachment sheath 130. Attachment sheath 130 is an additional sheath positioned external to internal sheath 110, and includes a distal portion surrounding both internal supporting member sheath portion 111 and guidewire sheath portion 113, and a proximal portion for guidewire sheath portion 113. This configuration is similar to the wire with wire loop portion 124 as shown in FIG. 4B, but instead of a wire, a sheath is used. An advantage of using a sheath is that the sheath can include a balloon 134 at a distal end thereof, with an inflation lumen 136 through attachment sheath 130, as shown in FIG. 4E. These types of balloons are known in the art and may be used to enhance apposition of device 10 to the inner wall of the vessel.

Reference is now made to FIGS. 5A-5F, which are cross-sectional illustrations of device 10 and delivery system 100 in various stages of deployment. As shown in FIG. 5A, initially, flexible inverting portion 15 of extending segment 14 sits inside supporting segment 12. Supporting segment 12 is shown in an expanded configuration, and includes a supporting member 30 surrounding supporting segment 12. Supporting member 30 is shown herein as an external supporting member. It should be readily apparent that this configuration occurs only after removal of supporting segment sheath 102. Internal supporting member 32 is held in an unexpanded configuration by internal supporting member sheath portion 111 of internal sheath 110, which is positioned distal to supporting segment 12 and to flexible inverting portion 15. Extension portion 114 of guidewire 104 is also positioned within internal supporting member sheath portion 111 of internal sheath 110, and further includes a stopper 116 in the vicinity of distal end 112. Stopper 116 is designed to hold internal supporting member 32 in place as internal sheath 110 is removed. Guidewire sheath portion 113 surrounds a portion of guidewire 104 which is proximal to bent distal portion 112 of guidewire 104, and extends proximally through bypass segment 16 to a proximal end of delivery system 100. Guidewire sheath portion 113 is attached to internal supporting member sheath portion 111 at attachment area 115. Guidewire 104 also extends through bypass segment 16 within guidewire sheath portion 113 of internal sheath 110 to the proximal end of delivery system 100.

As shown in FIG. 5B, guidewire 104 and guidewire sheath portion 113 of internal sheath 110 may be pulled proximally, causing internal supporting member sheath portion 111 of internal sheath 110, extension portion 114 of guidewire 104, stopper 116 and internal support member 32 to move proximally into supporting segment 12. As shown in FIG. 5C, this proximal motion continues until flexible inverting portion 15 is almost completely straightened out. During this proximal motion, guidewire sheath portion 113 and the portion of guidewire 104 which is within guidewire sheath portion 113 are configured to move proximally through bypass segment 16 while internal supporting member sheath portion 111 moves proximally within the vessel. This motion is made possible by the fact that these two portions are separatable proximal to attachment area 115 and it can occur until attachment area 115 reaches an intersection of bypass segment 16 and extended extending portion 14. As shown in FIG. 5D, guidewire 104 is then held in place, while internal sheath 110 is pushed distally to release internal supporting member 32. Stopper 116 holds internal supporting member 32 in place while internal sheath 110 is pushed distally. Alternatively, internal supporting member 32 may be expanded with a balloon included on guidewire 104 or by any other method. Internal supporting member 32 may be comprised of a metal, polymer, or any other substantially rigid material which can provide support in a vessel. As shown in FIG. 5E, guidewire 104 may then be pushed distally, internal supporting member sheath portion 111 and guidewire sheath portion 113 are brought together via linking element 120, and then guidewire 104 and internal sheath 110 may be pulled together proximally through bypass segment 16. Finally, as shown in FIG. 5F, once delivery system 100 is removed via bypass segment 16 device 10 remains in place in the vessel.

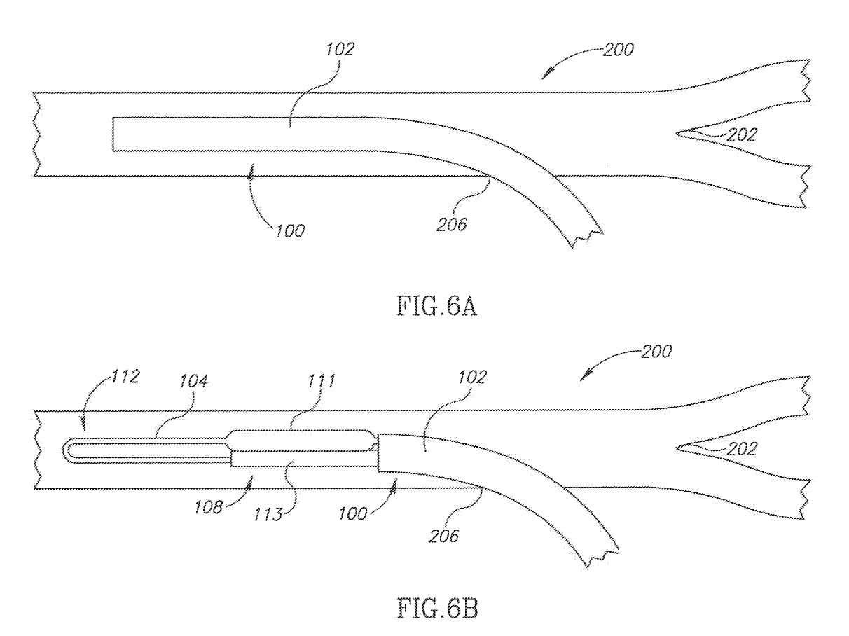

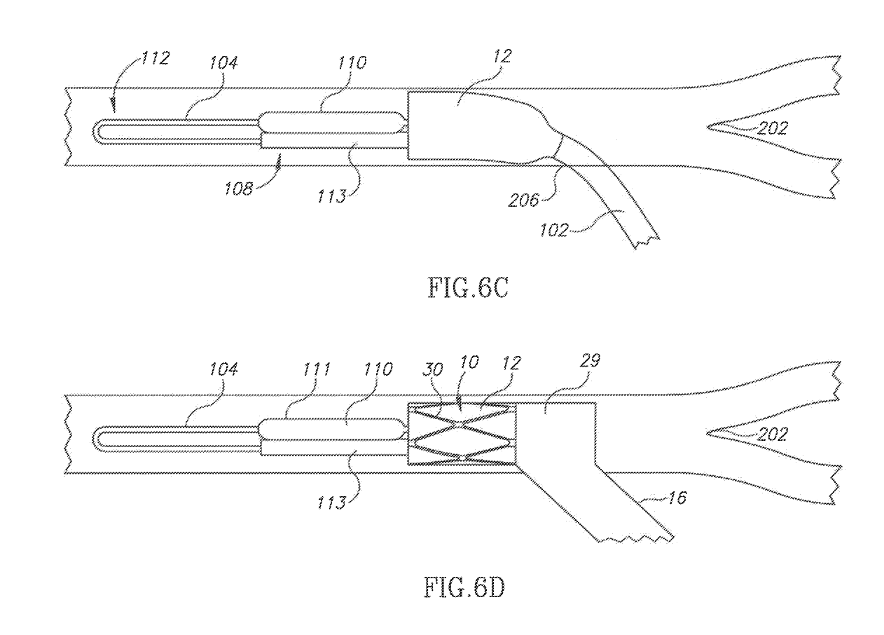

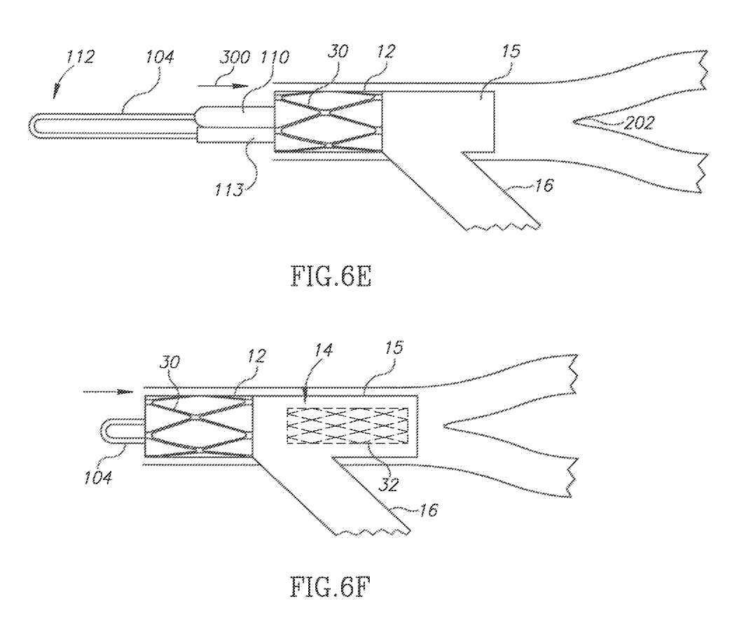

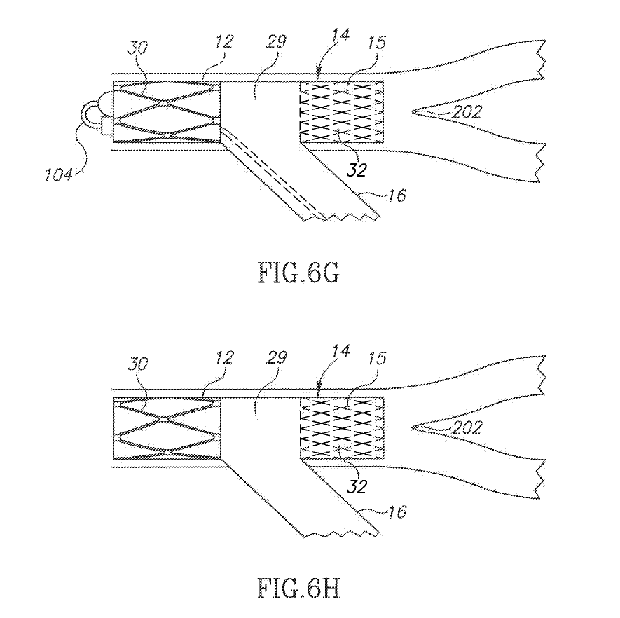

Reference is now made to FIGS. 6A-6H, which are illustrations of the various stages of deployment as described with reference to FIGS. 5A-5F, shown in the vessel 200. As shown in FIG. 6A, delivery system 100 with device 10 positioned therein is introduced into vessel 200 at an incision area 206. Delivery system 100 is positioned upstream from bifurcation. As shown in FIG. 6B, supporting segment sheath 102 is pulled back proximally, exposing bent distal end 112 of guidewire 104 and internal sheath 110. As shown in FIG. 6C, supporting segment sheath 102 is pulled further proximally, thus releasing supporting segment 12. As shown in FIG. 6D, supporting segment 12 with supporting member 30 anchors device 10 in place in within vessel 200. As shown in FIG. 6E, guidewire 104 and internal sheath 110 are pulled in a proximal direction, as shown by arrow 300, and flexible inverting portion 15 begins to assume its uninverted configuration. Once flexible inverting portion 15 is in position, as shown in FIG. 6F, internal supporting member 32 is expanded, as shown in 6G, by holding guidewire 104 in place while pushing internal sheath 110 distally. Finally, as shown in FIG. 6H, remaining portions of delivery system 100 are removed from vessel 200, leaving device 10 in place, wherein supporting segment 12 and extending segment 14 are in vessel 200, and wherein bypass segment 16 is external to vessel 200 for diverting blood flow away from the obstructed vessel while still allowing blood to flow through the unobstructed branch vessel.

Extending segment 14 is not limited to the configuration described herein. For example, extending segment 14 may have a flap configuration wherein in a first configuration the flap is folded in and adjacent to supporting segment 12 and in a second configuration the flap is extended proximally into the vessel. Other configurations are possible as well and are included within the scope of the present invention.

By using a system, device and method such as the ones described herein, it is possible to perform a percutaneous minimally invasive bypass procedure by directly accessing the vessel only through an incision in the vessel and anchoring the device therein, without the need for suturing or other complicated anastomoses. This procedure can serve as an alternative to surgical bypass, which is an extensive procedure requiring long hospital stays and associated with high risk. In some cases, when the surgery is considered extremely high risk, this may provide the only alternative for saving a limb.

1. A device for positioning in a vessel, the device comprising:
a supporting segment comprised of a flexible material having a substantially tubular configuration and a supporting member comprised of a substantially rigid material, said supporting segment comprising a supporting segment proximal end and a supporting segment distal end;
an extending segment having a substantially tubular configuration comprising a flexible inverting portion comprised of a flexible material, said flexible inverting portion having a flexible inverting portion proximal end and a flexible inverting portion distal end; and an internal supporting member comprised of a rigid material, said internal supporting member having an internal supporting member proximal end and an internal supporting member distal end, said internal supporting member proximal end attached to said flexible inverting portion proximal end at an attachment area, wherein in an initial configuration, said flexible inverting portion is inverted such that said flexible inverting portion proximal end is distal to said flexible inverting portion distal end and said internal supporting member is distal to said flexible inverting portion, and wherein in a deployed configuration, said flexible inverting portion is un-inverted such that said flexible inverting portion proximal end is proximal to said flexible inverting portion distal end and said internal supporting member is positioned within said flexible inverting portion; and
a bypass segment comprised of a flexible material having a substantially tubular configuration comprising a bypass segment proximal end and a bypass segment distal end, wherein said supporting segment, said extending segment and said bypass segment are in fluid communication with one another, and wherein said supporting segment proximal end, said extending segment distal end and said bypass segment proximal end are connected at a connecting area.

2. The device of claim 1, wherein said supporting segment, said extending segment and said bypass segment are comprised of the same material.

3. The device of claim 1, wherein said supporting member is comprised of a metal.

4. The device of claim 1, wherein said supporting member is an external supporting member and is positioned external to said flexible material of said supporting segment.

5. The device of claim 1, wherein said supporting member is sandwiched between two layers of said flexible material of said supporting segment.

6. The device of claim 1, wherein said internal supporting member is comprised of a metal.

7. The device of claim 1, wherein said internal supporting member further comprises an additional layer of flexible material therein, wherein said internal supporting member surrounds said additional layer of flexible material.

8. The device of claim 1, wherein said supporting member extends over said connecting area.

9. The device of claim 1, wherein said supporting member extends over a portion of said bypass segment.

10. A method for performing a minimally invasive bypass procedure, the method comprising:
providing a device having a supporting segment, an extending segment, and a bypass segment, wherein each of said supporting, extending and bypass segments is in fluid communication with the others of said supporting, extending and bypass segments, said extending segment initially in an unextended configuration;
making an incision in a vessel to be treated;
inserting the supporting segment directly into the vessel through the incision and positioning the supporting segment in the vessel distal to the incision with the bypass segment positioned through the incision and out of the vessel;
anchoring the supporting segment into the vessel; and
extending said extending segment in a proximal direction such that the extending segment is positioned in the vessel proximal to the incision.

11. The method of claim 10, wherein the vessel to be treated is a portion of a vessel which is upstream of a bifurcation.

12. The method of claim 10, wherein a flexible inverting portion of said extending segment is initially in an inverted configuration and wherein said extending comprises uninverting said flexible inverting portion.

13. The method of claim 12, wherein said uninverting comprises providing a delivery system having a guidewire with a bent distal end and a proximal extension portion extending from said bent distal end, and wherein said proximal extension portion is attached to said extending segment; and pulling the guidewire in a proximal direction so as to push said extending segment in a proximal direction, thereby uninverting said extending segment.

14. The method of claim 10, wherein said extending segment comprises a flexible inverting portion having an initial inverted configuration and an internal supporting member having an uninverted configuration, and wherein said extending comprises uninverting the flexible inverting portion while the internal supporting member retains its uninverted configuration, such that following said uninverting, said internal supporting member is positioned inside said flexible inverting portion.

15. A delivery system for delivery of a graft to a vessel, the delivery system comprising:
a guidewire having a guidewire proximal end and a guidewire distal end, wherein said guidewire proximal end is positionable outside of a body and wherein said guidewire distal end is configured to enter the body at an incision site, said guidewire distal end having a bent configuration, said guidewire further comprising a proximal extension portion extending proximally from said guidewire distal end;
an internal sheath having a first portion for enclosing a first segment of the graft and a second portion for enclosing at least a portion of said guidewire and extending proximally to a point outside of the body, wherein said proximal extension portion of said guidewire is partially enclosed by said first portion of said internal sheath and is removably attached to the first segment of the graft located within said internal sheath, said internal sheath movable with respect to said guidewire;
a stopper attached to said proximal extension portion of said guidewire for holding the graft in place while said internal sheath is removed; and
an external sheath for enclosing a second member of the graft and extending proximally to a point outside of the body, said external sheath movable with respect to said internal sheath and said guidewire.

16. The delivery system of claim 15, wherein said first segment of the graft enclosed within said internal sheath is an extending segment, wherein in an initial configuration, a proximal end of said extending segment is distal to a distal end of said extending segment, and wherein in a final configuration, said proximal end of said extending segment is proximal to said distal end of said extending segment.

17. The delivery system of claim 15, further comprising a linking member for separatably placing said first portion and said second portion of said internal sheath together.

18. A device for positioning in a vessel, the device comprising:
a supporting segment configured to be placed directly through an incision in a vessel and to be anchored into the vessel in an area distal to the incision;
an extending segment, wherein in a first configuration said extending segment is positioned within said supporting segment and in a second configuration said extending segment is extended proximally so as to be anchored into the vessel in an area proximal to the incision; and
a bypass segment in fluid communication with said supporting segment and said extending segment, said bypass segment positioned through the incision and outside of the vessel.

19. The device of claim 18, wherein said first configuration is an inverted configuration and said second configuration is an un-inverted configuration.

20. The device of claim 18, wherein said supporting segment comprises a flexible material and a substantially rigid material.

21. The device of claim 18, wherein said extending segment comprises a flexible material and a substantially rigid material.
